(19) United States
(12) Patent Application Publication
(10) Pub. No.: US 2024/0131266 A1
(43) Pub. Date: Apr. 25, 2024

(54) SELF-ASPIRATING SYRINGE SYSTEMS, CARTRIDGES, AND METHODS

(71) Applicant: Eli Lilly and Company, Indianapolis, IN (US)

(72) Inventor: Eric Magnus CHELLQUIST, Douglassville, PA (US)

(21) Appl. No.: 18/547,460

(22) PCT Filed: Feb. 24, 2022
(86) PCT No.: PCT/US2022/017680

§ 371 Date: Aug. 21, 2023

Related U.S. Application Data
(60) Provisional application No. 63/153,420, filed on Feb. 25, 2021.

(51) Int. Cl. A61M 5/28; A61M 5/32

(52) U.S. Cl. CPC A61M 5/285; A61M 5/32; A61M 2207/10

(57) ABSTRACT

A cartridge is configured to receive an injectable drug. The cartridge includes a body, and the body includes a proximal end portion having a proximal opening, a distal end portion having a distal opening, and a bore coupling the proximal opening and the distal opening. The cartridge further includes a pierceable septum coupled to the distal opening and a plunger movably carried within the bore. A volume is disposed in the bore between the pierceable septum and the plunger. The volume has a reduced pressure, and the reduced pressure is less than atmospheric pressure. A stopper is detachably coupled to the distal opening.

### CROSS-REFERENCE TO RELATED APPLICATION

This application claims the benefit of U.S. Provisional Application No. 63/153,420, filed Feb. 25, 2021.

### BACKGROUND

The present disclosure relates to syringe systems for delivering injectable drugs to subjects, and, in particular, self-aspirating syringe systems and cartridges having reduced-pressure volumes that facilitate self-aspiration.

Injectable drugs are commonly provided in multiple-dose or single-dose vials, and such agents are dispensed from the vials to hypodermic syringes for subsequent delivery to patients. More specifically, this process typically begins by holding the syringe with the needle pointing upwardly. The syringe's plunger is then retracted to fill the syringe with air, specifically a volume of air equal to the volume of the injectable drug to be administered to a patient. The needle of the syringe is then inserted through a stopper coupled to the opening of a vial, and the plunger is advanced to deliver the air from the syringe to the vial. Next, the vial and the syringe are inverted, and the needle's tip is held in the injectable drug. The plunger is then retracted to fill the syringe with the injectable drug, and the needle is removed from the vial. The syringe is then ready to deliver the injectable drug to a patient.

For some individuals, such as diabetics, the above process is relatively difficult or inconvenient. In other settings, such as high-throughput contract pharmacies, the above process is overly time consuming. Accordingly, it would be desirable to provide improved syringe systems.

### SUMMARY

According to an embodiment of the present disclosure, a cartridge is configured to receive an injectable drug. The cartridge includes a body, and the body includes a proximal end portion having a proximal opening, a distal end portion having a distal opening, and a bore coupling the proximal opening and the distal opening. The cartridge further includes a pierceable septum coupled to the distal opening and a plunger movably carried within the bore. A volume is disposed in the bore between the pierceable septum and the plunger. The volume has a reduced pressure, and the reduced pressure is less than atmospheric pressure. A stopper is detachably coupled to the distal opening.

According to another embodiment of the present disclosure, a syringe system is configured for delivering an injectable drug to a subject. The syringe system includes a cartridge, a vial, a transfer device, and a delivery needle. The cartridge includes a body having a bore, a plunger movably carried within the bore, and a volume disposed in the bore. The volume has a reduced pressure, and the reduced pressure is less than atmospheric pressure. The vial carries the injectable drug. The transfer device is configured to deliver the injectable drug from the vial to the bore of the cartridge. The delivery needle is configured to be coupled to the distal end portion of the cartridge and deliver the injectable drug from the cartridge to the subject.

According to yet another embodiment of the present disclosure, a method of manufacturing a cartridge, the cartridge being configured to receive an injectable drug, includes: providing a body including a distal opening, a proximal opening, and bore extending between the distal opening and the proximal opening; positioning a plunger in the bore; coupling a stopper to the proximal opening; reducing a pressure in the bore to less than atmospheric pressure; and sealing the distal opening.

According to yet another embodiment of the present disclosure, a method of delivering an injectable drug from an internal chamber of a vial to an internal bore of a cartridge, the internal bore initially having a reduced pressure volume, includes venting the internal chamber of the vial such that the injectable drug is exposed to atmospheric pressure; coupling a transfer device to the vial; coupling the transfer device to the cartridge, the transfer device thereby facilitating fluid communication between the internal chamber of the vial and the internal bore of the cartridge; and permitting the injectable drug to flow from the internal chamber of the vial, through the transfer device, and into the internal bore of the cartridge.

### BRIEF DESCRIPTION OF THE DRAWINGS

The above-mentioned and other advantages and objects of this disclosure, and the manner of attaining them, will become more apparent, and the disclosure itself will be better understood, by reference to the following description of embodiments of the disclosure taken in conjunction with the accompanying drawings, wherein:

FIG. 1 is a side sectional view of a cartridge for a syringe system according to an embodiment of the present disclosure.

FIG. 2 is a flow diagram illustrating a method of delivering an injectable drug to a subject according to an embodiment of the present disclosure.

FIG. 3 is a side sectional view of a vial carrying an injectable drug and the cartridge of FIG. 1; the vial and the cartridge are provided according to the method illustrated in FIG. 2.

FIG. 4 is a side sectional view of a transfer needle being coupled to the vial according to the method illustrated in FIG. 2.

FIG. 5 is a side sectional view of a needle hub being coupled to the transfer needle according to the method illustrated in FIG. 2.

FIG. 6 is a side sectional view of the cartridge being coupled to the needle hub, the transfer needle, and the vial according to the method illustrated in FIG. 2.

FIG. 7 is a side sectional view of an injectable drug flowing from the vial to the cartridge according to the method illustrated in FIG. 2.

FIG. 8 is a side sectional view of the vial, the transfer needle, a nut, and a stopper being uncoupled from the cartridge according to the method illustrated in FIG. 2.

FIG. 9 is a side sectional view of a delivery needle and an actuation rod being coupled to the cartridge according to the method illustrated in FIG. 2.

FIG. 10 is a side sectional view of the cartridge coupled to the vial via a flexible conduit according to an alternative of the method illustrated in FIG. 2.

FIG. 11 is a flow diagram illustrating a method of manufacturing a cartridge according to an embodiment of the present disclosure.

FIG. 12 is a side sectional view of a plunger being coupled to a body of the cartridge according to the method illustrated in FIG. 11.

FIG. 13 is a side sectional view of a stopper, a nut, and a pierceable septum being coupled to the body of the cartridge according to the method illustrated in FIG. 11.

FIG. 14 is a side sectional view of the cartridge positioned in a pressure reduction chamber according to the method illustrated in FIG. 11.

FIG. 15 is a side sectional view of the septum being coupled to the cartridge according

to the method illustrated in FIG. 11.

FIG. 16 is a side sectional view of a seal being coupled to the cartridge according to the method illustrated in FIG. 11.

Corresponding reference characters indicate corresponding parts throughout the several views. Although the drawings represent embodiments of the present invention, the drawings are not necessarily to scale, and certain features may be exaggerated or omitted in some of the drawings in order to better illustrate and explain the present invention.

### DETAILED DESCRIPTION

Syringe systems according to the present disclosure dispense one or more injectable drugs, which may also be referred to as medications or therapeutic agents. Such injectable drugs may include, for example, vaccines, antibodies, insulins, insulin analogs, insulin derivatives, GLP-1 receptor agonists, epinephrine, anaesthetics, analgesics, steroids, imaging/contrast agents, radiopharmaceutical agents, or any other drug that is capable of delivery by systems according to the present disclosure. Syringe systems according to the present disclosure are operated in a manner generally as described herein by a user (for example, a healthcare professional, a caregiver, or another person) to deliver one or more injectable drugs to a subject (for example, a patient, the user himself or herself, another person, or an animal).

Any directional references used with respect to any of the Figures, such as right or left, up or down, or top or bottom, are intended for convenience of description, and does not limit the present disclosure or any of its components to any particular positional or spatial orientation.

Self-aspirating syringe systems, cartridges, and methods according to embodiments of the present disclosure provide one or more of various advantages. For example, systems and cartridges according to embodiments of the present disclosure may be relatively simple to operate and inexpensive to manufacture.

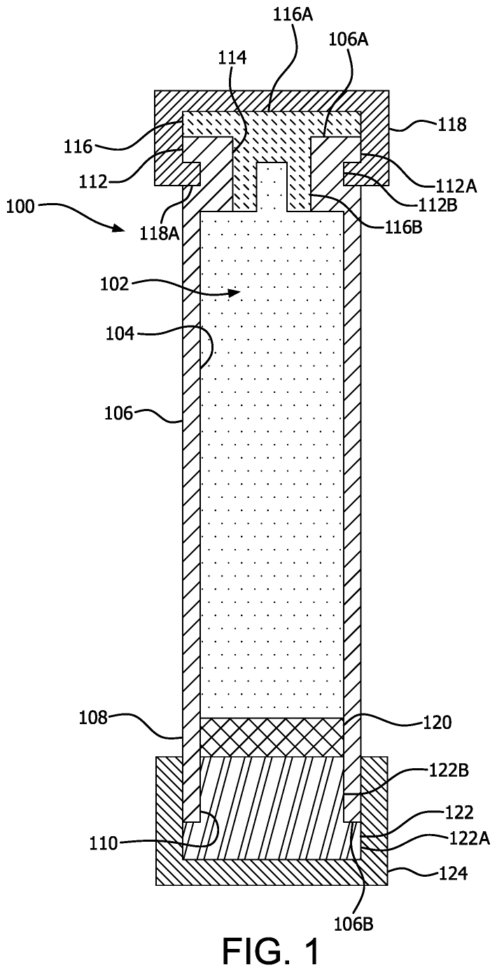

FIG. 1 illustrates a cartridge 100 for a syringe system (shown elsewhere) according to an embodiment of the present disclosure. The cartridge 100 is illustrated in an initial configuration, or a configuration in which the cartridge 100 is provided to a user prior to delivering an injectable drug to a subject. More specifically, the cartridge 100 is illustrated as having a reduced-pressure volume 102 within an internal bore 104 of a main body 106. The volume 102 has a reduced pressure relative to atmospheric pressure, and the reduced pressure facilitates aspirating an injectable drug from a vial (shown elsewhere) and delivering the injectable drug to the bore 104 of the cartridge 100. These aspects are described in further detail below.

The reduced-pressure volume 102 may comprise a gas (for example, air) or the reduced-pressure volume 102 may be a vacuum. The reduced-pressure volume 102 may have any of various pressures that are less than atmospheric pressure (that is, 1 atm (101.325 kPa)), including, for example, 0.9 atm (91.193 kPa), 0.8 atm (81.060 kPa), 0.7 atm (70.928 kPa), 0.6 atm (60.795 kPa), 0.5 atm (50.663 kPa), 0.4 atm (40.530 kPa), 0.3 atm (30.398 kPa), 0.2 atm (20.265 kPa), 0.1 atm (10.133 kPa), or 0.0 atm (0.0 kPa). As described in further detail below, the pressure of the reduced-pressure volume 102 affects the volume of injectable drug that is aspirated from a vial with the volume at a higher pressure than the reduced-pressure volume 102, which may be, for example, at atmoshpheric pressure, and transferred to the bore 104 of the cartridge 100.

With continued reference to FIG. 1, the cartridge 100 also includes various other components that facilitate initially sealing the bore 104 from the external environment, transferring an injectable drug to the cartridge 100, and delivering the injectable drug to a subject. More specifically, the body 106 of the cartridge 100 includes a proximal end portion 108 that has a proximal opening 110 and a distal end portion 112 that has a distal opening 114. The bore 104 couples and extends between the proximal opening 110 and the distal opening 114. At the distal end portion 112, a pierceable septum 116 is coupled to the distal opening 114, and a seal 118 covers the pierceable septum 116. The distal end portion 112 may be configured to receive a needle assembly. In one example, the distal end portion 112 defines an outer radial flange 112A and a reduced neck region 112B that is adjacent the flange 112A, such as shown in FIG. 1. At the proximal end portion 108, a plunger 120 is movably carried within the bore 104, and a vacuum stopper 122 is detachably coupled to the distal opening 114 such that the plunger 120 is disposed between the stopper 122 and the reduced-pressure volume 102. The proximal end portion 108 also includes a fastener, more specifically a vacuum nut 124, that secures the stopper 122 between the nut 124 and the plunger 120.

In one embodiment, as shown in FIG. 1, the pierceable septum 116 is constructed to include a distal head portion 116A that is disposed against the distal facing surface 106A defined by the body 106 and a proximal reduced-area portion 116B extending proximally from the distal head portion 116A and sized to engage the inner wall of the body 106 that defines the distal end opening 112. In one embodiment, as shown in FIG. 1, the seal 118 is constructed having a cup shape with an inner radial lip 118A proximally spaced from an inner axial wall of the seal 118, whereby the inner radial lip 118A is sized to extend within and fit within the reduced neck region 112B of the body's distal end portion 112, and the interior space of the seal 118 capturing the distal head portion 116A of the septum 116 and the thickness of the flange 112A. In other embodiments, the seal 118 threadably couples to the body 106. In one embodiment, as shown in FIG. 1, the stopper 122 is constructed to have a proximal head portion 122A that is disposed against the proximal facing surface 106B or edge defined by the body 106 and a distal reduced-area portion 122B extending distally from the proximal head portion 122A and sized to engage the inner wall defining the inner bore 104 at the proximal end opening 110. In one embodiment, as shown in FIG. 1, the nut 124 is constructed to have a cup shape having an inner diameter sized to fit over the outer diameter of the proximal head portion 122A of the stopper 122 and the outer diameter of the body 106.

The components of the cartridge 100 may be constructed of various appropriate materials. The body 106 may be constructed of, for example, glass or plastic. The pierceable septum 116, the plunger 120, the stopper 122, and/or the nut 124 may be constructed of, for example, plastic. The seal 118 may be constructed of, for example, metal.

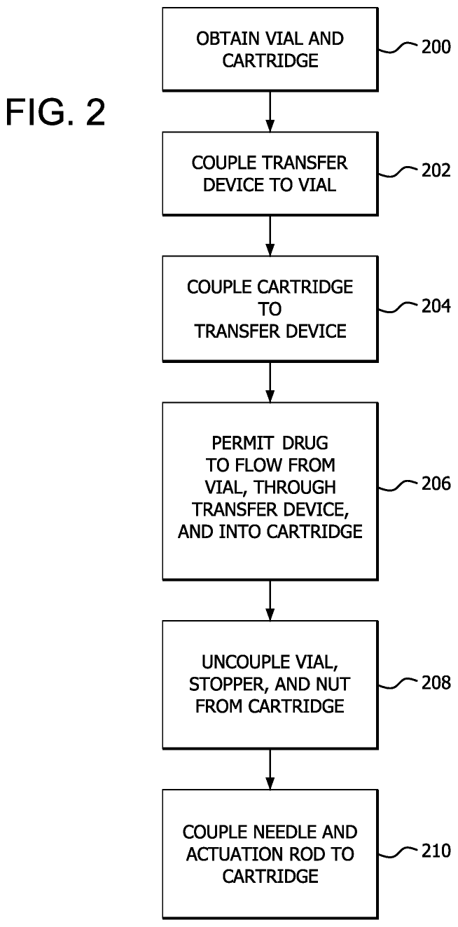

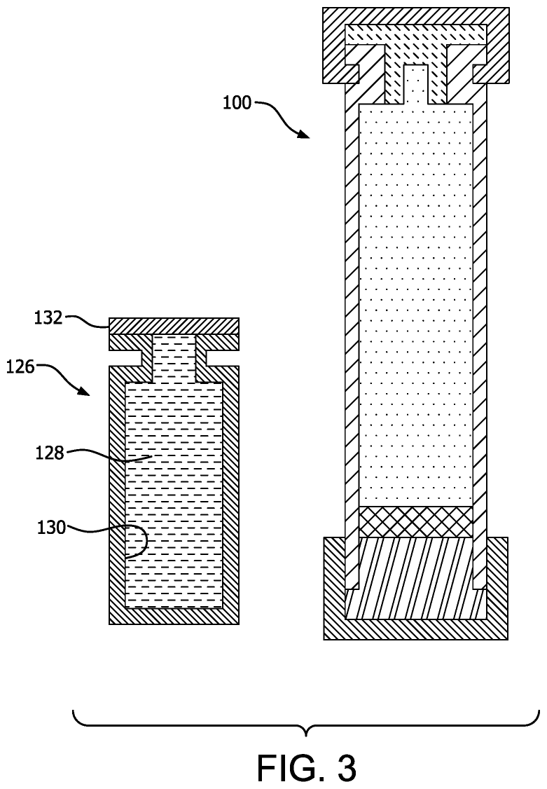

FIG. 2 is a flow diagram illustrating a method of using a syringe system to deliver an injectable drug to a subject, according to an embodiment of the present disclosure. FIGS. 3-10 illustrate various actions associated with the method.

The method begins, as shown at block 200 of FIG. 2 and as shown in FIG. 3, by providing a drug vial 126, or a vial 126 carrying an injectable drug 128 in an internal chamber 130, and the cartridge 100 in its initial configuration (that is, having the reduced-pressure volume 102 as shown in FIG. 1). The method continues by piercing a septum 132 of the vial 126 with a vent filter (not shown) such that the injectable drug is exposed to atmospheric pressure.

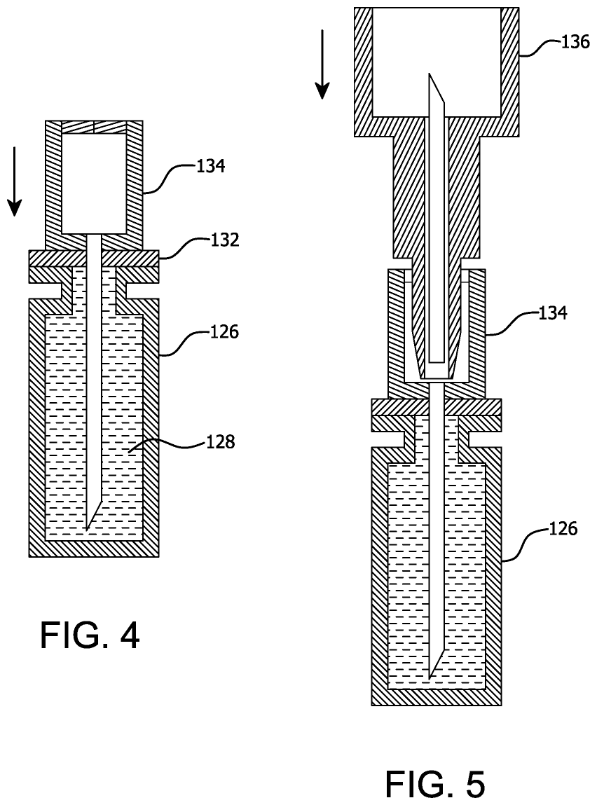

With the vent filter remaining coupled to the vial 126, the method continues, as shown at block 202, by coupling a transfer device to the vial 126. More specifically, and as shown in FIG. 4, the transfer device may include a transfer needle 134 that pierces the septum 132 of the vial 126 to access the injectable drug 128. As shown in FIG. 5, the transfer device may further include a needle hub 136 that couples to the transfer needle 134.

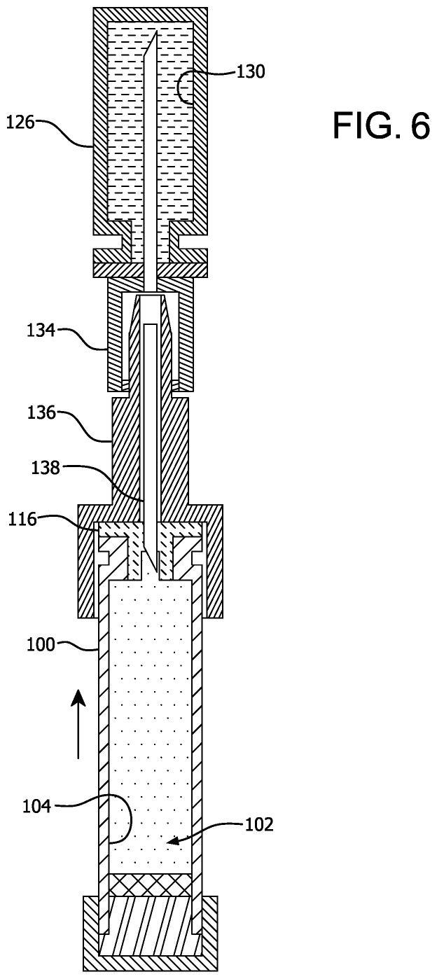

Next, after the seal 118 (shown elsewhere) is removed from the distal end portion 112 of the cartridge 100 and, as shown at block 204, the cartridge 100 is coupled to the transfer device and the vial 126. More specifically, and as shown in FIG. 6, the cartridge 100 is coupled to an end of the needle hub 136 opposite the transfer needle 134 and the vial 126. As a result, a transfer cannula 138 of the needle hub 136 pierces the septum 116, and the transfer cannula 138 thereby accesses the internal bore 104 and reduced-pressure volume 102 of the cartridge 100. As a result, the transfer device facilitates fluid communication between the internal chamber 130 of the vial 126 and the internal bore 104 of the cartridge 100. The method continues at block 206 by permitting the injectable drug 128 to flow from

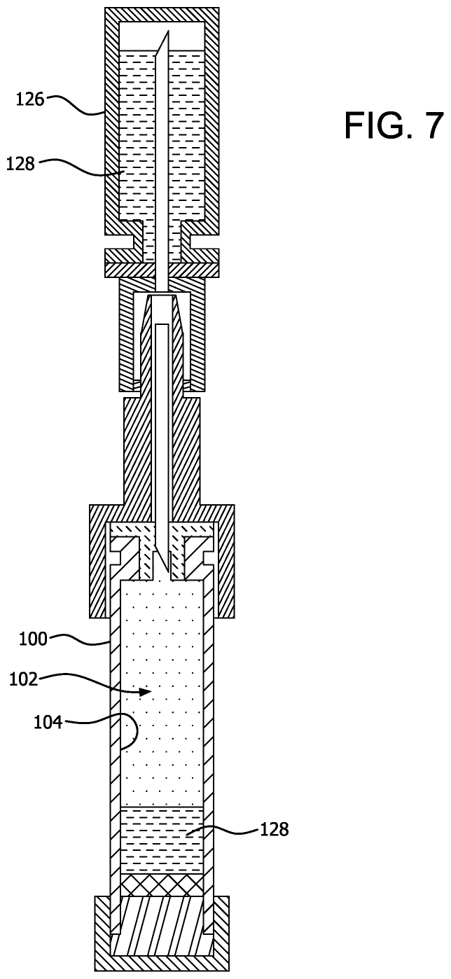

the internal chamber 130 of the vial 126, through the transfer device, and into the internal bore 104 of the cartridge 100. More specifically, and referring to FIG. 7, because the injectable drug 128 in the vial 126 is exposed to atmospheric pressure and the reduced-pressure volume 102 is present in the bore 104, the injectable drug 128 flows from the vial 126 to the cartridge 100. Flow of the injectable drug 128 terminates when any gas in the reduced-pressure volume 102 is sufficiently compressed and reaches atmospheric pressure or, if the reduced-pressure volume 102 is a vacuum, when the cartridge 100 is completely filled by the injectable drug 128.

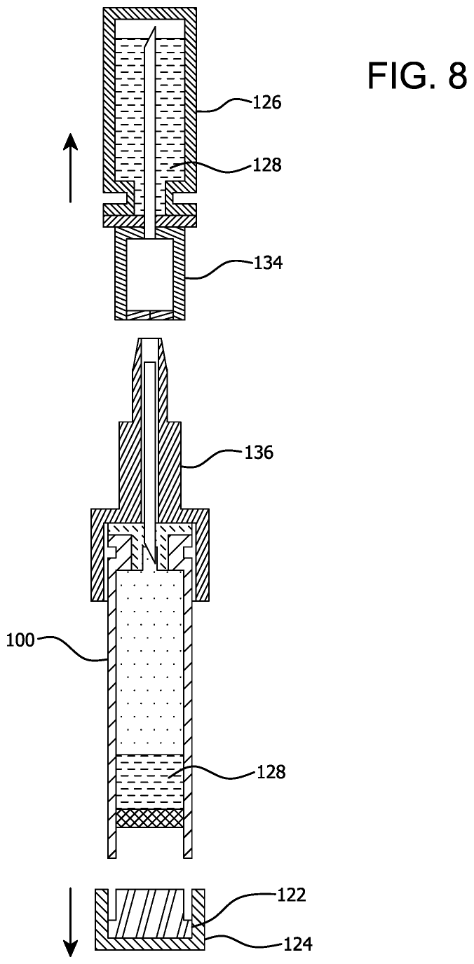

The method continues at block 208 by uncoupling the vial 126, the stopper 122, and the nut 124 from the cartridge 100. More specifically, and as shown in FIG. 8, the vial 126 and the transfer needle 134 are uncoupled from the needle hub 136. The vial 126 may be discarded if it carries a single dose of the injectable drug 128 or used with additional cartridges 100 if it carries multiple doses of the injectable drug 128.

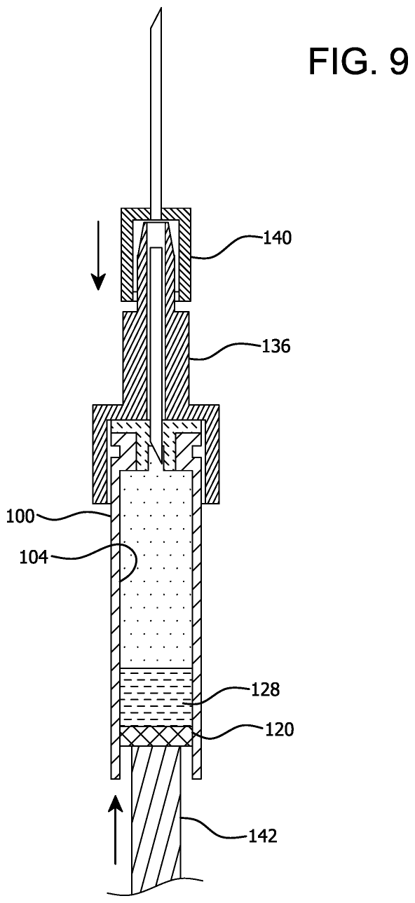

Next, and as shown at block 210 and in FIG. 9, a delivery needle 140 is coupled to the needle hub 136 and an actuation rod 142 is coupled to the plunger 120 (for example, via a threaded post carried by the plunger 120—not shown). A user may advance the rod 142 and the plunger 120 to expel any gas remaining in the bore 104 of the cartridge 100, and the syringe system is then ready to deliver the injectable drug 128 to a subject.

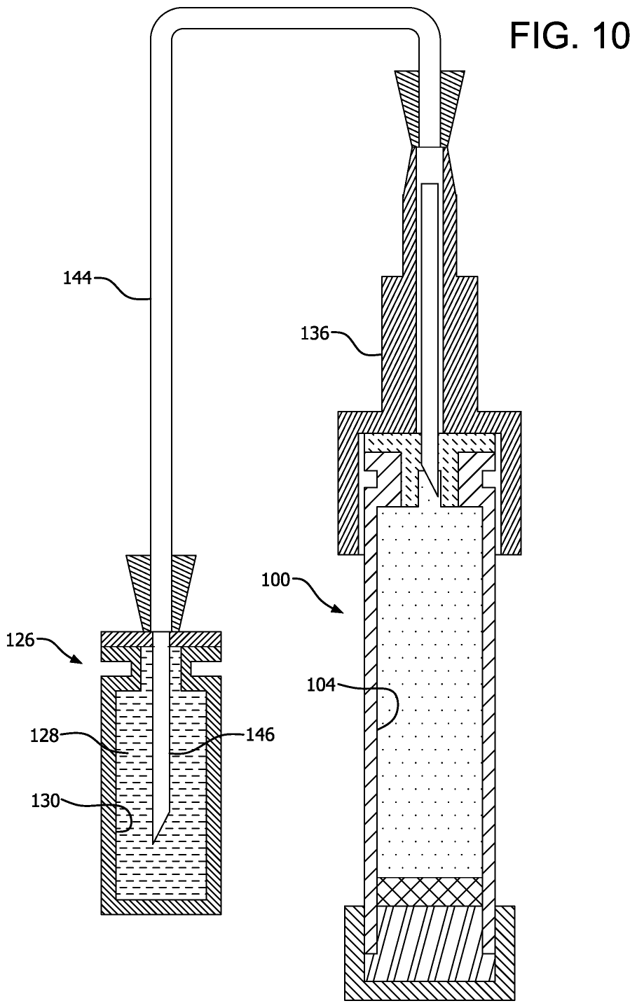

The method described above may be modified in various manners. For example, a different transfer device may be used to deliver the injectable drug 128 from the vial 126 to the cartridge 100. As a specific example and referring to FIG. 10, the transfer device may include tubing or a flexible conduit 144 that couples to a transfer needle 146 and the needle hub 136 and, as a result, facilitates fluid communication between the internal chamber 130 of the vial 126 and the internal bore 104 of the cartridge 100.

The cartridge 100 may alternatively be used in connection with other methods. For example, the cartridge 100 may be used to aspirate a liquid from a subject. More specifically, the cartridge 100 may be used to aspirate a liquid from the lung of a subject. In such a method, the cartridge 100 may be used with an actuation needle hub (not shown) to pierce the lung of the subject with the external needle of the hub. Subsequently, the internal needle of the hub may pierce the septum 116 of the cartridge 100 to facilitate aspirating the liquid into the cartridge 110.

As described briefly above, the initial pressure of the reduced-pressure volume 102 affects the volume of injectable drug that is aspirated from a vial and transferred to the bore 104 of the cartridge 100. More specifically, and based on the ideal gas law (if the temperature and amount of any gas in the reduced-pressure volume 102 remain constant), the amount of an injectable drug, Vₐ, transferred into the bore 104 of the cartridge 100 is as follows:

Vₐ=Vᵢ(1−(Pᵢ/P_{f}))

where:

Vᵢ is the initial volume of the reduced-pressure volume 102;

Pᵢ is the initial pressure of the reduced-pressure volume 102; and

P_{f} is the final pressure of the reduced-pressure volume 102 (that is, after transferring the injectable drug to the bore 104), typically atmospheric pressure.

Some syringe systems according to embodiments of the present disclosure may include various cartridges having different sizes and/or reduced-pressure volumes with different pressures, and a user may select a cartridge appropriate for delivering a specific volume of an injectable drug to a subject.

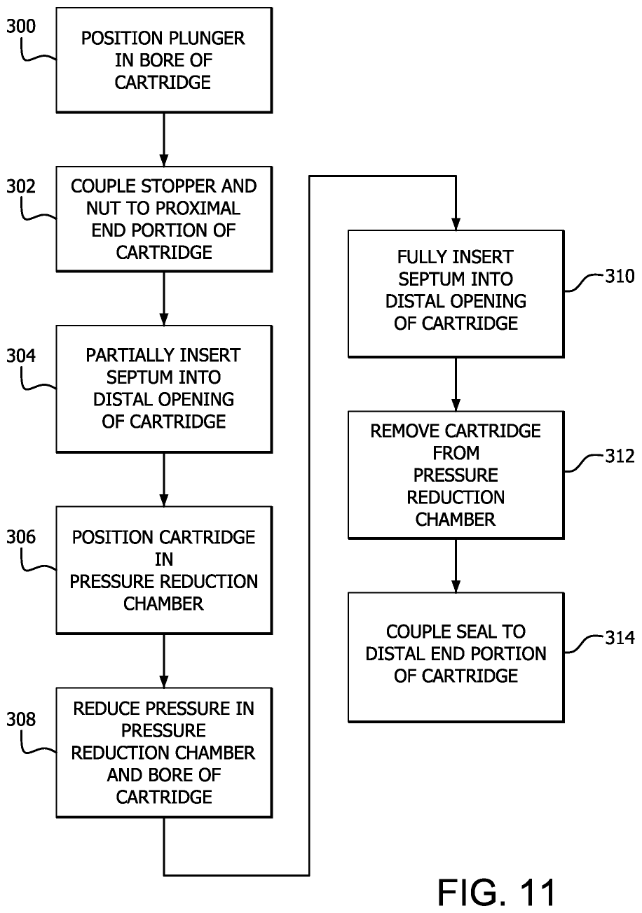

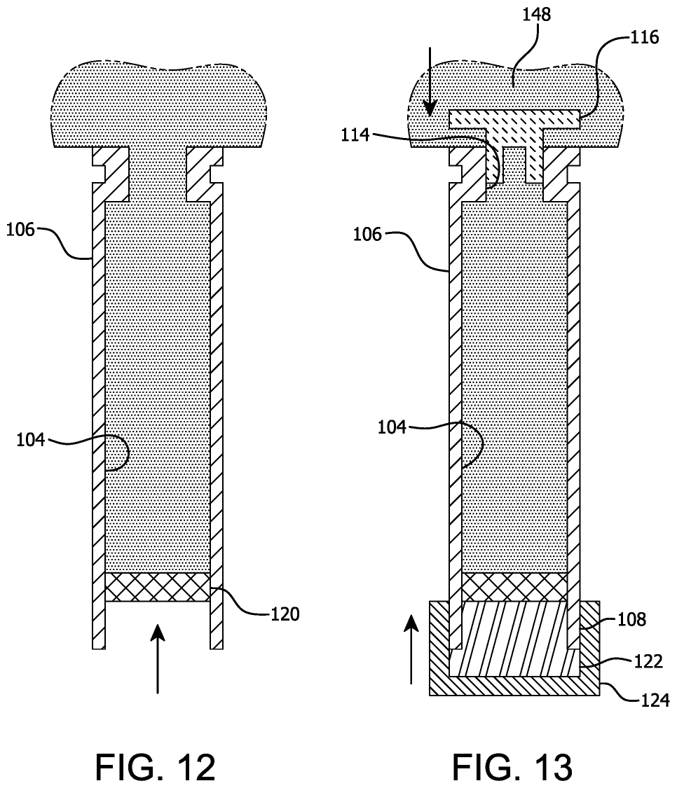

FIG. 11 is a flow diagram illustrating a method of manufacturing the cartridge 100 according to an embodiment of the present disclosure. FIGS. 12-16 illustrate various actions associated with the method.

The method begins as shown at block 300 by positioning the plunger 120 in bore 104 of the cartridge 100. More specifically, and as shown in FIG. 12, the plunger 120 is positioned in the bore 104 of the body 106 while the bore 104 is exposed to a gas, for example, air, at a non-reduced pressure, such as atmospheric pressure.

Next, and as shown at block 302 and in FIG. 13, the stopper 122 and the nut 124 are coupled to the proximal end portion 108 of the body 106. As shown at block 304 and in FIG. 13, the septum 116 is partially inserted in the distal opening 114 such that the bore 104 remains in fluid communication with the external environment 148 of the body 106.

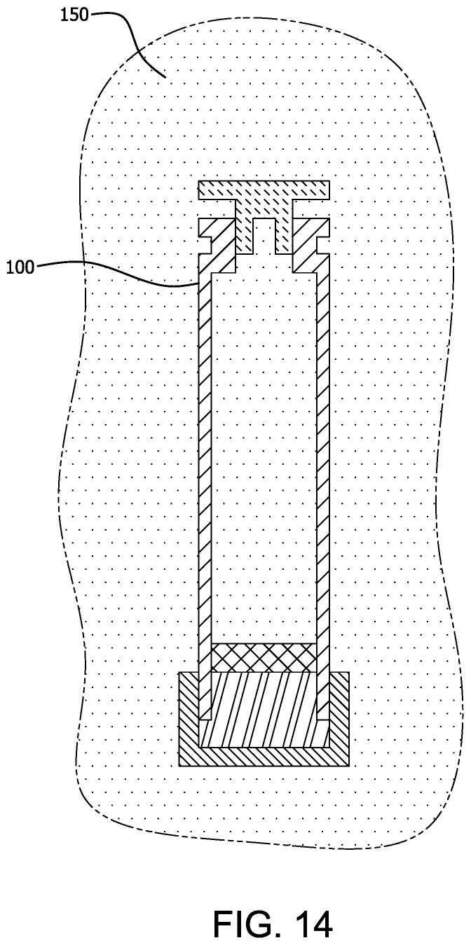

Next, and as shown at block 306 and in FIG. 14, the cartridge 100 is positioned in a vacuum or pressure reduction chamber 150. Then, and as shown at block 308, the pressure in the pressure reduction chamber 150 and the bore 104 of the cartridge 100 is reduced.

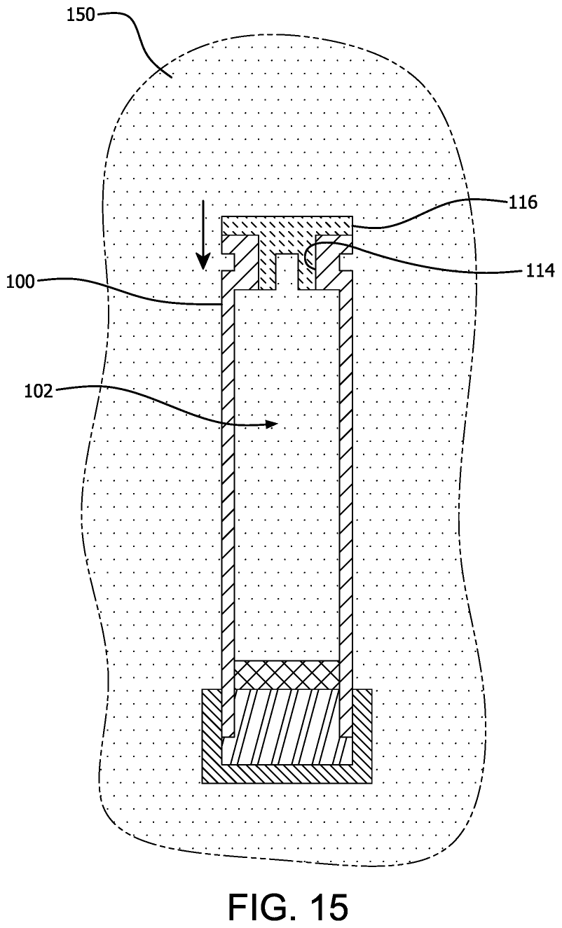

Next, and as shown at block 310 and in FIG. 15, the septum 116 is fully inserted into the distal opening 114 of the cartridge 100, thereby sealing the vacuum or reduced pressure of the vacuum or pressure reduction chamber 150 in the cartridge 100 as the reduced-pressure volume 102. Then, and as shown at block 312, the cartridge 100 is removed from the pressure reduction chamber 150.

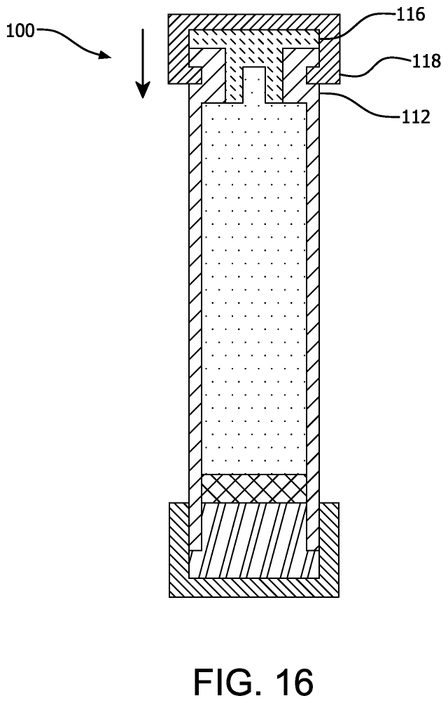

Finally, and as shown at block 314 and in FIG. 16, the seal 118 is coupled to the distal end portion 112 of the cartridge 100 and over the septum 116.

While this invention has been shown and described as having preferred designs, the present invention may be modified within the spirit and scope of this disclosure. This application is therefore intended to cover any variations, uses or adaptations of the invention using its general principles. Further, this application is intended to cover such departures from the present disclosure as come within known or customary practice in the art to which this invention pertains.

Various aspects are described in this disclosure, which include, but are not limited to, the following aspects:

1. A cartridge for receiving an injectable drug, comprising: a body comprising: a proximal end portion comprising a proximal opening; a distal end portion comprising a distal opening; a bore coupling the proximal opening and the distal opening; a pierceable septum coupled to the distal opening; a plunger movably carried within the bore; a volume disposed in the bore between the pierceable septum and the plunger, the volume having a reduced pressure, the reduced pressure being less than atmospheric pressure; and a stopper detachably coupled to the distal opening.

2. The cartridge of claim 1, wherein the volume comprises a gas having the reduced pressure.

3. The cartridge of any one of claims 1-2, further comprising a seal coupled to the distal end portion and covering the pierceable septum.

4. The cartridge of any one of claims 1-3, further comprising a nut detachably coupled to the proximal end portion and securing the stopper between the nut and the plunger.

5. The cartridge of any one of claims 1-4, wherein the plunger is configured to couple to an actuating rod upon detachment of the stopper from the body.

6. A syringe system for delivering an injectable drug to a subject, comprising: a cartridge, comprising: a body comprising a bore; a plunger movably carried within the bore; a volume disposed in the bore, the volume having a reduced pressure, the reduced pressure being less than atmospheric pressure; a vial carrying the injectable drug; a transfer device configured to deliver the injectable drug from the vial to the bore of the cartridge; and a delivery needle configured to be coupled to the distal end portion of the cartridge and deliver the injectable drug from the cartridge to the subject.

7. The syringe system of claim 6, wherein the body further comprises: a distal end

portion comprising a distal opening; a proximal end portion comprising a proximal opening, the proximal opening coupled to the distal opening by the bore; and wherein the cartridge further comprises a pierceable septum coupled to the distal opening.

8. The syringe system of claim 6, further comprising a distal seal coupled to the distal end portion and covering the pierceable septum.

9. The syringe system of any one of claims 7-8, wherein the transfer device comprises a transfer cannula configured to pierce the pierceable septum.

10. The syringe system of claim 6, wherein the body further comprises: a distal end portion comprising a distal opening; a proximal end portion comprising a proximal opening, the proximal opening coupled to the distal opening by the bore; and wherein the cartridge further comprises a stopper coupled to the proximal opening.

11. The syringe system of claim 6, wherein the cartridge further comprises a nut detachably coupled to the proximal end portion and securing the stopper between the nut and the plunger.

12. The syringe system of any one of claims 6-11, further comprising an actuation rod configured to couple to the plunger.

13. The syringe system of any one of claims 6-12, wherein the transfer device comprises a flexible conduit.

14. A method of manufacturing a cartridge, the cartridge configured to receive an injectable drug, the method comprising: providing a body comprising a distal opening, a proximal opening, and bore extending between the distal opening and the proximal opening; positioning a plunger in the bore; coupling a stopper to the proximal opening; reducing a pressure in the bore to less than atmospheric pressure; and sealing the distal opening.

15. The method of claim 14, further comprising, prior to reducing the pressure in the bore, partially inserting a septum into the distal opening such that the bore remains in fluid communication with an external environment of the body.

16. The method of claim 14, wherein sealing the distal opening comprises further inserting the septum into the distal opening and covering the septum with a seal.

17. The method of any one of claims 15-16, further comprising coupling a nut to the body and over the stopper.

18. The method of any one of claims 15-17, wherein reducing the pressure in the bore comprises positioning the body in a pressure reduction chamber.

19. The method of claim 14, further comprising, while the body is positioned in the pressure reduction chamber, inserting a septum into the distal opening to inhibit fluid communication between the bore and the pressure reduction chamber.

20. The method of claim 14, further comprising removing the body from the pressure reduction chamber, and wherein sealing the distal opening comprises, after removing the body from the pressure reduction chamber, covering the septum with a seal.

21. A method of delivering a injectable drug from an internal chamber of a vial to an internal bore of a cartridge, the internal bore initially having a reduced pressure volume, the method comprising: venting the internal chamber of the vial such that the injectable drug is exposed to atmospheric pressure; coupling a transfer device to the vial; coupling the transfer device to the cartridge, the transfer device thereby facilitating fluid communication between the internal chamber of the vial and the internal bore of the cartridge; and permitting the injectable drug to flow from the internal chamber of the vial, through the transfer device, and into the internal bore of the cartridge.

22. The method of claim 21, wherein coupling the transfer device to the vial comprises piercing a septum of the vial with a needle of the transfer device.

23. The method of any one of claims 21-22, wherein coupling the transfer device to the cartridge comprises piercing a septum of the cartridge with a needle of the transfer device.

24. The method of any one of claims 21-23, after permitting the injectable drug to flow into the internal bore of the cartridge, further comprising: decoupling the transfer device from the cartridge; coupling a needle to the cartridge; and delivering the injectable drug to a subject via the needle.

What is claimed is:

1. A cartridge for receiving an injectable drug, comprising:
a body comprising:
a proximal end portion comprising a proximal opening;
a distal end portion comprising a distal opening;
a bore coupling the proximal opening and the distal opening;
a pierceable septum coupled to the distal opening;
a plunger movably carried within the bore;
a volume disposed in the bore between the pierceable septum and the plunger, the volume having a reduced pressure, the reduced pressure being less than atmospheric pressure; and
a stopper detachably coupled to the distal opening.

2. The cartridge of claim 1, wherein the volume comprises a gas having the reduced pressure.

3. The cartridge of claim 1, further comprising a seal coupled to the distal end portion and covering the pierceable septum.

4. The cartridge of claim 1, further comprising a nut detachably coupled to the proximal end portion and securing the stopper between the nut and the plunger.

5. The cartridge of claim 1, wherein the plunger is configured to couple to an actuating rod upon detachment of the stopper from the body.

6. A syringe system for delivering an injectable drug to a subject, comprising:
a cartridge, comprising:
a body comprising a bore;
a plunger movably carried within the bore;
a volume disposed in the bore, the volume having a reduced pressure, the reduced pressure being less than atmospheric pressure;
a vial carrying the injectable drug;
a transfer device configured to deliver the injectable drug from the vial to the bore of the cartridge; and
a delivery needle configured to be coupled to the distal end portion of the cartridge and deliver the injectable drug from the cartridge to the subject.

7. The syringe system of claim 6, wherein the body further comprises:
a distal end portion comprising a distal opening;
a proximal end portion comprising a proximal opening, the proximal opening coupled to the distal opening by the bore;
and wherein the cartridge further comprises a pierceable septum coupled to the distal opening.

8. The syringe system of claim 7, further comprising a distal seal coupled to the distal end portion and covering the pierceable septum.

9. The syringe system of claim 7, wherein the transfer device comprises a transfer cannula configured to pierce the pierceable septum.

10. The syringe system of claim 6, wherein the body further comprises:
a distal end portion comprising a distal opening;
a proximal end portion comprising a proximal opening, the proximal opening coupled to the distal opening by the bore;
and wherein the cartridge further comprises a stopper coupled to the proximal opening.

11. The syringe system of claim 10, wherein the cartridge further comprises a nut detachably coupled to the proximal end portion and securing the stopper between the nut and the plunger.

12. The syringe system of claim 6, further comprising an actuation rod configured to couple to the plunger.

13. The syringe system of claim 6, wherein the transfer device comprises a flexible conduit.

14. A method of manufacturing a cartridge, the cartridge configured to receive an injectable drug, the method comprising:
providing a body comprising a distal opening, a proximal opening, and bore extending between the distal opening and the proximal opening;
positioning a plunger in the bore;
coupling a stopper to the proximal opening;
reducing a pressure in the bore to less than atmospheric pressure; and
sealing the distal opening.

15. The method of claim 14, further comprising, prior to reducing the pressure in the bore, partially inserting a septum into the distal opening such that the bore remains in fluid communication with an external environment of the body.

16. The method of claim 15, wherein sealing the distal opening comprises further inserting the septum into the distal opening and covering the septum with a seal.

17. The method of claim 15, further comprising coupling a nut to the body and over the stopper.

18. The method of claim 15, wherein reducing the pressure in the bore comprises positioning the body in a pressure reduction chamber.

19. The method of claim 18, further comprising, while the body is positioned in the pressure reduction chamber, inserting a septum into the distal opening to inhibit fluid communication between the bore and the pressure reduction chamber.

20. The method of claim 19, further comprising removing the body from the pressure reduction chamber, and wherein sealing the distal opening comprises, after removing the body from the pressure reduction chamber, covering the septum with a seal.

21. A method of delivering an injectable drug from an internal chamber of a vial to an internal bore of a cartridge, the internal bore initially having a reduced pressure volume, the method comprising:
venting the internal chamber of the vial such that the injectable drug is exposed to atmospheric pressure;
coupling a transfer device to the vial;
coupling the transfer device to the cartridge, the transfer device thereby facilitating fluid communication between the internal chamber of the vial and the internal bore of the cartridge; and
permitting the injectable drug to flow from the internal chamber of the vial, through the transfer device, and into the internal bore of the cartridge.

22. The method of claim 21, wherein coupling the transfer device to the vial comprises piercing a septum of the vial with a needle of the transfer device.

23. The method of claim 21, wherein coupling the transfer device to the cartridge comprises piercing a septum of the cartridge with a needle of the transfer device.

24. The method of claim 21, after permitting the injectable drug to flow into the internal bore of the cartridge, further comprising:
decoupling the transfer device from the cartridge;
coupling a needle to the cartridge; and
delivering the injectable drug to a subject via the needle.
